(19) United States
(12) Patent Application Publication
(10) Pub. No.: US 2012/0101340 A1
(43) Pub. Date: Apr. 26, 2012

(54) Dolphin Blade/Peri-laryngoscope

(76) Inventor: Albert Mburu, Grand Prairie, TX (US)

(21) Appl. No.: 12/908,722

(22) Filed: Oct. 20, 2010

(51) Int. Cl. A61B 1/267

(52) U.S. Cl. 600/189; 600/188

(57) ABSTRACT

A laryngoscope may be a device to view the vocal chords of a patient and may include a channel wall to define a channel, an input port to the channel being connected to the channel wall to allow a user to view the vocal chords of the patient, and output port to the channel being connected on an opposing side of the channel wall to allow the user to view the vocal chords of the patient. The input port may include a first light reflecting device, and the output port may include a second light reflecting device. The input port may include a first inclined end, and the outlet port may include a second inclined end.

### FIELD OF THE INVENTION

The present invention relates to an airway device and more particularly to a laryngoscope.

### BACKGROUND

The present invention relates to a laryngoscope configured to facilitate the alignment of the oropharyngeal, laryngeal, and tracheal axes of the airway of an individual in the supine position prior to induction of anesthesia and placement of an endotracheal tube. The present invention also relates to a modification to enable visualization of the oral/pharyngeal cavities and larynx.

### BACKGROUND OF THE RELATED ART

One of the most important tasks of an anesthesiologist is the management of the patient's airway during the administration of anesthesia. This task may be performed during emergency situations such as CPR or cardiac arrest, ENT procedures or in the operating room. However, intubation is a crucial procedure in the medical field and the use of the laryngoscope has generally remained unchanged for over a century. The patient has their necks stretched and the person administrating the laryngoscope should manipulate the laryngoscope in order to view the vocal chords. First, the mouth is opened then, using the laryngoscope (typically the curved Macintosh blade) the tongue is swept out of the way. The tip of the blade is inserted into the vallecula, and the person administrating the laryngoscope will visually locate the vocal chords and passes the endotracheal tube between the vocal chords. The vocal chords must be distinguished from the esophagus in order to prevent intubation into the wrong opening. The difficulty of this procedure is maneuvering safely around the anatomical corner or curve of almost 90° from the mouth to the larynx (the vocal chords). In conclusion, endotracheal intubation is a medical procedure in which an endotracheal tube is inserted through the oral cavity into the trachea to provide controlled or spontaneous ventilation and to protect the lungs from gastric acid contamination. This protection is provided by a balloon on the tip of the tube that is inflated when the tube is in the proper location above the carina. In order to maximize visualization of the larynx during endotracheal intubation, the anesthesiologist must place the patient's head and upper back in the “sniffing” position to properly align the oropharyngeal, laryngeal, and tracheal axes. The obese patient presents a challenge for proper alignment because of excess tissue in the upper back, neck, and oral pharynx.

Physiological changes occurring during pregnancy further complicate airway management in the pregnant patient. Intubation is also performed in emergency situations outside of the operating room such as the emergency rooms, hospital rooms, or at the scene of accidents.

### SUMMARY

A laryngoscope is a device to view the vocal chords of a patient and may include a channel wall to define a channel, an input port to the channel being connected to the channel wall to allow a user to view the vocal chords of the patient, and output port to the channel being connected on an opposing side of the channel wall to allow the user to view the vocal chords of the patient.

The input port may include a first light reflecting device, and the output port may include a second light reflecting device.

The input port may include a first inclined end, and the outlet port may include a second inclined end.

The first light reflecting device may include a prism, and the first light reflecting device may include a mirror.

The second light reflecting device may include a prism, and a second light reflecting device may include a mirror.

The output port may include a light.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a cross-sectional view of the laryngoscope device of the present invention;

FIG. 2 illustrates a perspective view of the laryngoscope device of the present invention;

FIG. 3 illustrates a front view of the laryngoscope device of the present invention;

FIG. 4 illustrates a back view of the laryngoscope device of the present invention;

FIG. 5 illustrates a cross-sectional view of the channel wall of the laryngoscope device of the present invention.

### DETAILED DESCRIPTION

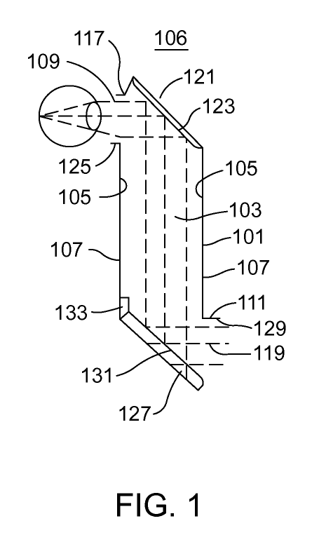

The present invention provides a user/operator with a view of the vocal chords of a patient without the need for a grueling neck extension and provides direct view. The present invention is particularly advantageous for obese patients, young patients or elderly patients. The present invention increases the success rate for intubations and reduces traumatic intubations, cardiac arrest and other complications from the use of prior art devices. The present invention provides smoother/safer anatomical appropriate curves in order to maneuver through the mouth and pharynx with minimal trauma or injury. FIG. 1 illustrates a laryngoscope device 100 to aid in the inspection of vocal chords of a patient in order to aid in the establishment of an airway for the patient. The laryngoscope device 100 may include a channel wall 101 which may be a cylindrical, oval or other shape wall and which may be formed from rigid or flexible material such as metal such as steel or durable aluminum alloy, plastic or other appropriate material and which may be sized to cooperate with the throat of a patient. The prisms or reflective surfaces of the present invention may be formed from glass or other appropriate materials. The channel wall 101 may define a light channel 103 in order to allow a visual inspection of the vocal chords by the user. The channel wall 101 may have an exterior channel surface 107 and a interior channel surface 105. The channel wall 101 may be connected to a input port 117 to allow access to the light channel 103 and may be connected to an output port 119 to allow access to the light channel 103.

The input port 117 may include a first end 109 which may include a first inclined wall 121 to mount a first light reflecting device 123 on the interior surface of the first inclined wall 121 and may include a first cylinder wall 125 to allow the user to access by viewing the first light reflecting device 123.

The output port 119 may include a second end 111 which may include a second inclined wall 127 to mount a second light reflecting device 129 on the interior surface of the second inclined wall 129 and may include a second cylinder wall 129 to allow the user to view the vocal chords of the patient. In addition, the output port 119 may include a light device 133 which may be powered by an internal battery (not shown) in order to provide sufficient light to view the vocal chords of the patient.

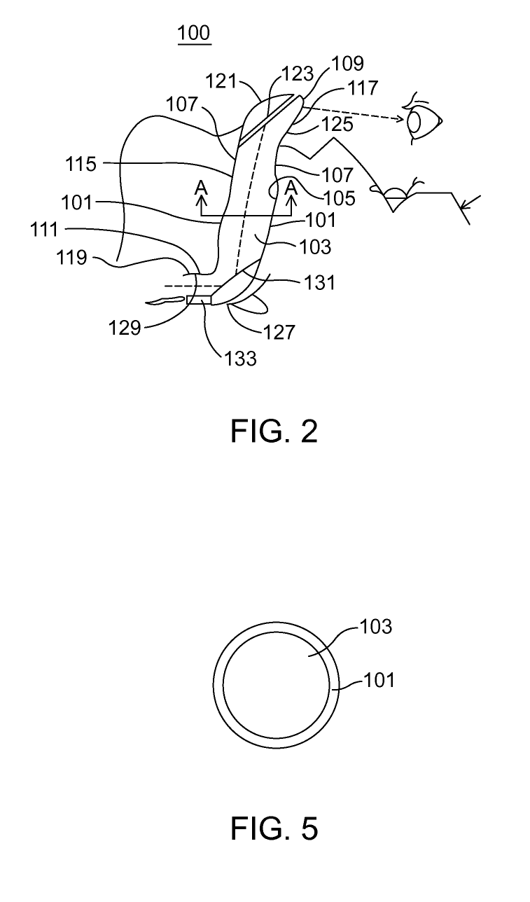

FIG. 2 illustrates a perspective view of the laryngoscope device 100 to aid in the inspection of vocal chords of a patient in order to aid in the establishment of an airway for the patient. The laryngoscope device 100 may include a channel wall 101 which may be a cylindrical, oval or other shape wall and which may be formed from rigid or flexible material such as metal, plastic or other appropriate material. The channel wall 101 may define a light channel 103 in order to allow a visual inspection of the vocal chords by the user. The channel wall 101 may have an exterior channel surface 107 and an interior channel surface 105. The channel wall 101 may be connected to an input port 117 to allow access to the light channel 103 and may be connected to an output port 119 to allow access to the light channel 103.

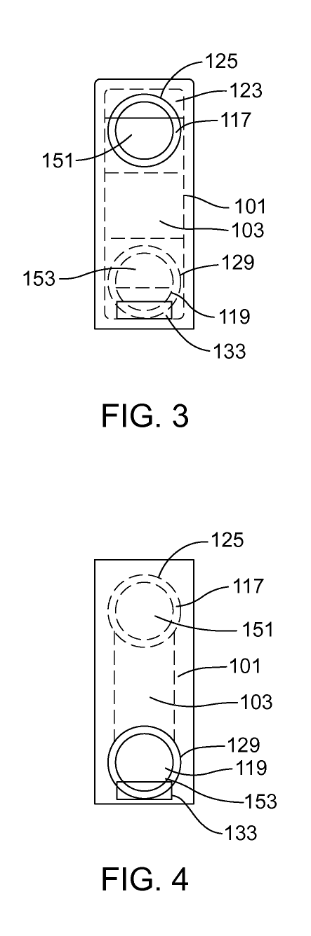

The input port 117 may include a first end 109 which may include a first inclined wall 121 to mount a first light reflecting device 123 on the interior surface of the first inclined wall 121 and may include a first cylinder wall 125 to allow the user to access by viewing the first light reflecting device 123. The input port 117 may optionally include a first lens 151 see FIG. 3 which may be detachably connected to the input port 117 in order to provide magnification/clarification of the vocal chords of the patient. The first lens 151 may be removed and replaced in order to provide different magnifications.

The output port 119 may include a second end 111 which may include a second inclined wall 127 to mount a second light reflecting device 131 on the interior surface of the second inclined wall 127 and may include a second cylinder wall 129 to allow the user to view the vocal chords of the patient. In addition, the output port 119 may include a light device 133 which may be powered by an internal battery (not shown) in order to provide sufficient light to view the vocal chords of the patient.

The output port 119 may optionally include a second lens 155 see FIG. 3 which may cooperate with the first lens 151 and which may be detachably connected to the output port 119 in order to provide magnification/clarification of the vocal chords of the patient. The second lens 155 may be removed and replaced in order to provide different magnifications.

FIG. 4 illustrates a back view of the laryngoscope device 100 to aid in the inspection of vocal chords of a patient in order to aid in the establishment of an airway for the patient. The laryngoscope device 100 may include a channel wall 101 which may be a cylindrical, oval or other shape wall and which may be formed from rigid or flexible material such as metal, plastic or other appropriate material. The channel wall 101 may define a light channel 103 in order to allow a visual inspection of the vocal chords by the user. The channel wall 101 may have an exterior channel surface 107 and a interior channel surface 105 see FIG. 1. The channel wall 101 may be connected to an input port 117 to allow access to the light channel 103 and may be connected to an output port 119 to allow access to the light channel 103.

The input port 117 may include a first cylinder wall 125 to allow the user to access by viewing the first light reflecting device 123.

The output port 119 may include a second light reflecting device 129 on the interior surface of the second inclined wall 127 and may include a second cylinder wall 129 to allow the user to view the vocal chords of the patient. In addition, the output port 119 may include a light device 133 which may be powered by an internal battery (not shown) in order to provide sufficient light to view the vocal chords of the patient.

FIG. 3 illustrates a front view of the laryngoscope device 100 to aid in the inspection of vocal chords of a patient in order to aid in the establishment of an airway for the patient. The laryngoscope device 100 may include a channel wall 101 which may be a cylindrical, oval or other shape wall and which may be formed from rigid or flexible material such as metal, plastic or other appropriate material. The channel wall 101 may define a light channel 103 in order to allow a visual inspection of the vocal chords by the user. The channel wall 101 may have an exterior channel surface 107 and a interior channel surface 105 see FIG. 1. The channel wall 101 may be connected to an input port 117 to allow access to the light channel 103 and may be connected to an output port 119 to allow access to the light channel 103.

The input port 117 may include a first cylinder wall 125 to allow the user to access by viewing the first light reflecting device 123.

The output port 119 may include a second light reflecting device 129 on the interior surface of the second inclined wall 127 see FIG. 1 and may include a second cylinder wall 129 to allow the user to view the vocal chords of the patient. In addition, the output port 119 may include a light device 133 which may be powered by an internal battery (not shown) in order to provide sufficient light to view the vocal chords of the patient.

FIG. 5 illustrates a cross section of the channel wall 101 and illustrates the channel 103.

The first light reflecting device and the second light reflecting device may be a prism or a mirror.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) A laryngoscope device to view the vocal chords of a patient, comprising:
a channel wall to define a channel;
an input port to the channel being connected to the channel wall to allow a user to view the vocal chords of the patient;
an output port to the channel being connected on an opposing side of the channel wall to allow the user to view the vocal chords of the patient;
wherein the input port includes a first light reflecting device and wherein the output port includes a second light reflecting device.

2) A laryngoscope device to view the vocal chords of a patient as in claim 1, wherein the input port includes a first inclined end.

3) A laryngoscope device to view the vocal chords of a patient as in claim 1, wherein the output port includes a second inclined end.

4) A laryngoscope device to view the vocal chords of a patient as in claim 1, wherein the first light reflecting device includes a prism.

5) A laryngoscope device to view the vocal chords of a patient as in claim 1, wherein the first reflecting device includes a mirror.

6) A laryngoscope device to view the vocal chords of a patient as in claim 1, wherein the second light reflecting device includes a prism.

7) A laryngoscope device to view the vocal chords of a patient as in claim 1, wherein the second light reflecting device includes a mirror.

8) A laryngoscope device to view the vocal chords of a patient as in claim 1, wherein the output port includes a light.
